(19) United States
(12) Patent Application Publication
(10) Pub. No.: US 2011/0268686 A1
(43) Pub. Date: Nov. 3, 2011

(54) COSMETIC COMPOSITION INCLUDING AN ACANTHUS EXTRACT, AND USE OF ACANTHUS IN A COSMETIC HAIR-CARE COMPOSITION

(75) Inventor: Christel Fiorini-Puybaret, Toulouse (FR)

(73) Assignee: PIERRE FABRE DERMO-COSMETIQUE, BOULOGNE (FR)

(21) Appl. No.: 13/138,054

(22) PCT Filed: Dec. 18, 2009
(86) PCT No.: PCT/FR2009/052610

§ 371 Date: Jun. 28, 2011

(30) Foreign Application Priority Data
Dec. 29, 2008 (FR) 0807481

(51) Int. Cl. A61K 8/97; A61Q 5/00

(52) U.S. Cl. 424/74

(57) ABSTRACT

The present invention relates to a new advantageous utilisation of acanthus in the cosmetic field, and more especially in a haircare composition for creating and/or maintaining the curling effect.

The invention relates to an extract of acanthus and to its use in a cosmetic composition, preferably in a haircare composition intended to create and/or maintain the curling effect.

The acanthus, or Acanthus sp., is a large, attractive, hardy plant belonging to the Acanthaceae family. It is mainly found in Mediterranean Europe and in North Africa. It is usually grown as an ornamental plant. Its highly decorative leaves were much used by the Greeks as the model for decorative friezes ornamenting Corinthian-capital columns. The fresh leaves and roots have been used since antiquity for their medicinal properties. Since antiquity, its sap has had the reputation of being a stimulating tonic. It was prescribed by Pliny and Dioscorides for treating wounds (emollient properties), and dysentery, diarrhoea and fever (contains mucilages) (LOUKIS and PHILIANOS, 1980—Phytochemical investigation of Acanthus molis L. Fitoterapia, 51 (4), 183-186). Via the oral route it was recommended as a diuretic and anti-dysentery agent. Topically it was reputed to be soothing and emollient (burns, fractures, mouth problems). Nowadays its use is relatively limited; it is still used in homeopathy for treating cough.

The acanthus is a hardy herbaceous plant measuring 30 to 80 cm in height, having a very long, creeping rhizome of whitish colour which may reach a diameter of 3 cm and which has numerous rootlets. Its habit is that of a spreading clump which may reach a width of 90 cm. The stems are round and strong.

The leaves are very large, slightly soft, of an attractive glossy dark-green colour, cut and deciduous; the leaves are simple, opposite, deeply lobed, dentate and carried on fleshy stems. The leafstalk is long.

When the leafstalk is pressed, the leaf releases a translucent, viscous “juice”, to which the benefits of the plant are attributed. This mucilage, which is also present in the other parts of the plant such as the roots, flowers and stems, is, as are most plant gums and exudates, made up of compounds formed by the condensation of carbohydrates (polysaccharides). Also found in the leaf are flavonoids (methoxylated flavones: hispidulin), phenolic acids (caffeic acids, chlorogenic acid), glycoproteins and amino acids.

The flowers are white and grouped on a tall upright flower spike (1 to 2 m). The flower spikes carry clusters of tubular flowers 5 to 6 cm in length, grouped by 2 s or 4 s.

The fruits are long, shiny, ovoid capsules, formed of 2 valves, releasing 2 to 4 fat seeds.

The Applicant has established a new advantageous utilisation of Acanthus mollis in the cosmetic, and more especially haircare, field wherein Acanthus mollis has, in unexpected and surprising manner, demonstrated a good ability to create tighter curls and to maintain the curling effect of the hair.

It is reasonable to envisage that the benefits of the present invention now being demonstrated by the inventors on the basis of Acanthus mollis, in view of the ease of supply of Acanthus mollis plant material in our country, may extend to the Acanthus genus or Acanthus sp.

Among the species of acanthus that are most reputed for their high mucilage content and/or that are readily available and abundant for cultivation for industrial purposes there may be mentioned especially: Acanthus mollis,

Acanthus ilicifolius, Acanthus ebracteatus, Acanthus montanus, Acanthus arboreus, Acanthus dioscoridis, Acanthus hungaricus, Acanthus sennii, Acanthus spinosus, Acanthus syriacus and Acanthus carduaceus.

The Acanthus species will be more preferably selected from the group consisting of Acanthus mollis, Acanthus ilicifolius, Acanthus ebracteatus and Acanthus montanus.

Even more preferably, the acanthus used in the context of the present invention is the species Acanthus mollis.

The present invention firstly relates to a new extract of acanthus having a fraction, by weight, of mucilages, expressed in terms of total reducing sugars, of between 1% and 80% inclusive, and advantageously more than 30%.

The present invention relates also to cosmetic compositions based on acanthus and more especially to cosmetic compositions comprising an extract of acanthus as defined hereinabove.

Finally, the present invention relates also to the use of acanthus in a cosmetic haircare composition intended for improving the creation of curls and maintaining the curling effect of hair.

As understood by the present invention, an “extract of acanthus” means:
the solution obtained after extraction of the Acanthus sp. plant with a solvent
or the juice obtained by pressing or extrusion.

The leaves, stems, flowers or roots or a mixture of those parts, preferably in fresh form, may be either extracted with a solvent or pressed to express the juice therefrom.

In a particular embodiment of the invention, the harvested parts of the plant are frozen before being stored.

Said extract of acanthus according to the present invention may be obtained as follows:
In the first case in point, the leaves, stems, flowers or roots or a mixture of those parts is/are preferably previously crushed and then extracted with a solvent which may be water or a mixture of water and water-miscible organic solvents such as an alcohol (methanol, ethanol etc.), a ketone (acetone) etc. The extraction is carried out in a plant/solvent ratio of between about 1/1 and about 1/20 inclusive and may be repeated 2 or 3 times. The temperature of the extraction solvent may be equal to ambient temperature or above, possibly reaching the boiling point of the solvent used. The time that the plant is in contact with the solvent is between about 30 mins. and about 72 hours inclusive.
In the second case in point, the juice of the leaves, stems, flowers or roots or a mixture of those parts, preferably in fresh form, may be obtained by pressing or by extrusion.

Solid/liquid separation is then carried out, the plant being separated from the solvent by filtration or centrifugation.

The filtrate obtained is concentrated in vacuo and at a temperature between ambient temperature and the boiling point, inclusive. The concentrate undergoes a sterilisation step by thermal treatment and a stabilisation step by addition of alcohol and is then dried by complete evaporation of the extraction solvent or kept as is in the form of a liquid extract. Drying of the final extract may be carried out by lyophilisation or by more conventional drying means known to the skilled person (nebulisation, oven, paddle dryer etc.).

The extract may be stabilised by addition of an antioxidant or a preservative such as, for example, ascorbic acid or citric acid in amounts of between about 0.05 and about 1 g, inclusive, per 100 g of dry extract.

The extract of acanthus is characterised chemically by its content of mucilages, of amino acids and of proteins (Van Hoist and Clarke, 1985—Quantification of Arabinogalactan-Protein in Plant Extracts by single Radial Gel Diffusion—Analytical Biochemistry 148, 446-450).

In the natural state, the plant contains a fraction, by weight, of mucilages which is between about 5% and about 10% inclusive.

In the context of the present invention there will preferably be used an extract of acanthus which has been purified to a greater or lesser extent.

The fraction, by weight, of mucilages of said extract, expressed in terms of total reducing sugars, is advantageously between about 1% and about 80%, inclusive, more advantageously between about 15% and about 80%, inclusive, and even more advantageously more than 30% (or between about 30% and about 80%, inclusive). In the case of an acanthus juice, the content of reducing sugars is usually of the order of about 5%.

Determination of the reducing sugars content may be carried out in any customary manner known to the skilled person.

The method of determining the reducing sugars content that is customarily used consists of a colorimetric determination of the reducing sugars content using 3,5-dinitrosalicylic (DNS) acid, relative to galactose. The results are expressed in terms of the reducing sugars percentage, relative to galactose. The method of determination is given hereinbelow:

Determination of the Total Reducing Sugars Content, Expressed in Terms of Galactose in Percent (w/w)

Preparation of reagents:
2N sodium hydroxide
DNS reagent:
   Sodium potassium ditartrate solution (solution 1):
   Dissolve 15 g of sodium potassium ditartrate in 25.0 ml of water. Stir until dissolution is complete.
   Dinitrosalicylic (DNS) solution (solution 2):
   Weigh 0.5 g of DNS into a 50 ml flask.
   Dissolve in 10 ml of 2N sodium hydroxide by gently heating with magnetic stirring at 30-35° C. for 1 hour.

Then add solution 1 to solution 2.

Heat gently (30-35° C.) with magnetic stirring until dissolution is complete (dissolution time: about 6 hours).

Preparation of the calibration series:
Reference 1: Dissolve 7.5 mg of galactose in 20 ml of water.
Reference 2: Dissolve 10.0 mg of galactose in 20 ml of water.
Reference 3: Dissolve 12.5 mg of galactose in 20 ml of water.
   Preparation of test solutions:

Dissolve 25.0 mg of extract, accurately weighed, in 20.0 ml of water. Perform 2 tests.

### Determination

Introduce into a series of 10-ml graduated tubes:

| | Ref1 | Ref2 | Ref3 | Test | Blank |
|---|---|---|---|---|---|
| Reference solutions (ml) | 1.0 | 1.0 | 1.0 | / | / |
| Test solutions (ml) | / | / | / | 1.0 | / |
| Ultra-pure water (ml) | 1.0 | 1.0 | 1.0 | 1.0 | 2.0 |
| DNS reagent (ml) | 1.0 | 1.0 | 1.0 | 1.0 | 1.0 |

Stir and then place in a water bath at 95° C. for 5 minutes.

Cool for about 10 mins. in an ice bath and make up to 10 ml with water.

Measure the absorbance of the various solutions at 500 nm against the blank.

### Calculation

Form the calibration curve and from it deduce the concentration of total reducing sugars (Q_{TRS}), expressed in terms of galactose, of the test solutions.

The content of total reducing sugars (C_{TRS}) in terms of percent (w/w) of the extract is given by the following formula:

$${C}_{TRS} ( \% ) = \frac{{Q}_{TRS} \times 100 \times 20}{We 2}$$

where Q_{TRS} is in mg/ml
We2 is in mg (sample weight in mg)

The present invention relates also to a cosmetic composition based on acanthus as active ingredient and at least one cosmetically acceptable excipient.

The acanthus is preferably in the form of an extract having a fraction, by weight, of mucilages, expressed in terms of total reducing sugars, of between 1% and 80% inclusive.

The cosmetic composition according to the present invention advantageously comprises an amount of dry extract of acanthus, by way of active ingredient, of between 0.010 g and 2 g, inclusive, and more preferably between 0.10 g and 0.70 g, inclusive, per 100 g of said composition.

The cosmetic composition according to the present invention may advantageously be in the form of any galenic form normally used in the cosmetic field for topical application. Preferably, the topical form may especially be in the form of: shampoo, balm, gel, lotion, mousse, spray, cream.

A distinction is accordingly made between products that are formulated for rinsing off and others that are not.

In one particular embodiment of the present invention, said amount of dry extract of acanthus is between 0.3 and 1 g, inclusive, and preferably equal to about 0.5 g, per 100 g of cosmetic composition intended to be rinsed off after its application.

In another particular embodiment of the invention, said amount of dry extract of acanthus is between about 0.1 and 0.3 g, inclusive, per 100 g of cosmetic composition which will not be rinsed off after its application to the hair.

The extract of acanthus may also be associated with other active agents such as avocado oil having nutritive properties.

The cosmetic composition according to the invention additionally comprises customary cosmetically compatible excipients.

Customary excipients compatible with the cosmetic haircare composition may be any excipient among those known to the skilled person for obtaining a cosmetic composition for topical application in the forms as described hereinbefore.

The cosmetic composition according to the invention may especially comprise additives and formulation aids such as surfactants of the emulsifying, cleaning, foaming etc. type, complexing agents, thickening agents, gel-forming agents, stabilisers, preservatives—including antimicrobials and antioxidants -, conditioners, acidifying agents, alkalinising agents, emollients, solvents, colourants, perfumes.

The composition may comprise other compounds that are useful in conditioning the hair such as, for example, colouring agents or shine-providing agents.

The present invention relates finally to the use of acanthus as a cosmetic agent in a cosmetic composition, and more especially in a haircare composition. Advantageously, the present invention relates to the use of acanthus as an active ingredient in a cosmetic haircare composition for improving or facilitating the formation of curls and/or maintaining the curling effect of the hair.

Acanthus has a curling effect and improves maintenance of the curls. The best results of the curling effect were noted when the acanthus is in the form of an extract of acanthus having a fraction, by weight, of mucilages, expressed in terms of total reducing sugars, of between 1% and 80%, inclusive.

More preferably, the present invention relates to the use of acanthus as active ingredient in a cosmetic haircare composition for improving the formation of curls and/or maintaining the curling effect of the hair, characterised in that the acanthus is in the form of an extract of acanthus having a fraction, by weight, of mucilages, expressed in terms of total reducing sugars, of between 1% and 80%, inclusive, and even more preferably of more than 30%.

Said extract of acanthus used in the context of the present invention additionally contains amino acids and proteins.

Finally, the acanthus will advantageously be selected from the group consisting of the following species: Acanthus mollis, Acanthus ilicifolius, Acanthus ebracteatus, Acanthus montanus, Acanthus arboreus, Acanthus dioscoridis, Acanthus hungaricus, Acanthus sennii, Acanthus spinosus, Acanthus syriacus and Acanthus carduaceus.

The compositions according to the present invention restore shape and body to curly, wavy or frizzy hair.

The effectiveness of the extract of acanthus on curls of the hair is reflected in:
the formation of tighter curls of hair (significantly shorter locks of hair, optimised curling effect);
better hold of the curls of hair (curls relax less quickly).

The following preparations and compositions are mentioned by way of illustrative, non-limiting examples.

### EXAMPLES OF PREPARATION OF THE PLANT EXTRACT

### Example 1

### Preparation of an Extract of Acanthus

The frozen fresh leaves of Acanthus mollis L. are crushed and then extracted with 7 volumes of water at 80° C. for 48 hours, with stirring.

The plant is separated from the solvent by filtration.

The filtrate obtained is concentrated in vacuo at a temperature of 80° C. to 30% of dry extract. The concentrate is sterilised at 121° C. for 20 mins. and stabilised by addition of ethanol.

The final extract is standardised to 70% of dry extract by addition of maltodextrin.

The extract obtained contains about 35 g of total reducing sugars, expressed in terms of galactose, per 100 g of dry extract.

### Example 2

### Preparation of an Extract of Acanthus

The frozen fresh whole leaves of Acanthus mollis are crushed and then introduced into an extruder in order to collect their juice. The juice is filtered off, with suction, in order to remove any plant debris, sterilised by heating at 121° C. for 20 mins. and then stabilised by addition of ethanol. After evaporation of alcohol, the stabilised alcoholic product is frozen and then dried by lyophilisation.

The extract obtained contains about 50 g of total reducing sugars, expressed in terms of galactose, per 100 g of dry extract.

### EXAMPLE OF COSMETIC COMPOSITIONS BASED ON ACANTHUS

### Example 1

### Balm to be Rinsed Off

| DRY ACANTHUS EXTRACT | 0.3 to 1 | g |
|---|---|---|
| AVOCADO OIL | 1.0 | g |
| CETEARYL ALCOHOL/CETEARETH 33 | 8.0 | g |
| CETEARYL ALCOHOL | 2.0 | g |
| AMODIMETHICONE SOLUTION | 8.0 | g |
| BEHENTRIMONIUM CHLORIDE | 3.0 | g |
| POLYQUATERNIUM 11 | 0.75 | g |
| BUTYLHYDROXYTOLUENE | 0.02 | g |
| EDTA 2 Na | 0.2 | g |
| NATURAL VINEGAR | 5.0 | g |
| Perfume(s) | q.s. | |
| Colourant(s) | q.s. | |
| Preservative(s) | q.s. | |
| PURIFIED WATER | q.s.p. 100.0 | g |
| DRY ACANTHUS EXTRACT | 0.3 to 1 | g |
| AVOCADO OIL | 0.1 | g |
| GUAR hydroxypropyltrimonium chloride | 0.5 | g |
| PROPYLENE GLYCOL | 1.0 | g |
| LAURETH SULFATE SODIUM | 9.5 | g |
| LAURYL BETAINE | 1.7 | g |
| LAURYL POLYGLUCOSIDE 50% | 5.0 | g |
| CETEARETH-60 MYRISTYL GLYCOL | 1.0 | g |
| EDTA SODIUM 2 Na | 0.2 | g |
| CETRIMONIUM CHLORIDE | 1.5 | g |
| Pearlescent agent | q.s. | |
| Preservative(s) | q.s. | |
| Perfume(s) | q.s. | |
| Colourant(s) | q.s. | |
| CITRIC ACID MONOHYDRATE | q.s.p pH = 5.0 | |
| PURIFIED WATER | q.s.p 100.0 | g |

### Example 2

### Shampoo

### Example 3

### Treatment, not for Rinsing Off

| DRY ACANTHUS EXTRACT | 0.1 to 0.3 | g |
|---|---|---|
| AVOCADO OIL | 0.5 | g |
| ACRYLIC POLYMER | 0.2 | g |
| NATURAL POLYSACCHARIDE | 0.1 | g |
| Cetylstearyl alcohol/ethoxylated cetylstearyl | 3.0 | g |
| alcohol (30 moles) | | |
| ETHYLHEXYL PALMITATE | 10.0 | g |
| CETEARYL ALCOHOL | 3.0 | g |
| AMODIMETHICONE SOLUTION | 2.0 | g |
| POLYQUATERNIUM 68 | 2.5 | g |
| CETRIMONIUM CHLORIDE | 0.5 | g |
| EDTA SODIUM 2 Na | 0.2 | g |
| Preservative(s) | q.s. | |
| Perfume(s) | q.s. | |
| PURIFIED WATER | q.s.p 100.0 | g |

### Biometrological Test on Locks of Hair

A cosmetic haircare efficacy study made it possible to assess the activity of a 1% aqueous solution of an acanthus extract obtained by the process described in Example 1 on maintaining curls in hair, compared to an untreated control. For that purpose, the locks are washed beforehand in standardised manner and then dried, also in standardised manner (drying for 18 hours under controlled temperature and humidity conditions: T°=21±1° C., relative humidity=50±5%). These locks are then immersed in the aqueous solution based on acanthus (treated group) or in water (control group) for 3 minutes.

The locks are then placed on a device allowing the creation of curls (rollers having a diameter of 16 mm) for 18 hours under the following temperature and humidity conditions: T°=21±1° C., relative humidity=50±5%; then 90 minutes at T°=25±1° C., relative humidity=75±5%.

The rollers are then taken out and the length of the locks is measured in the treated group and in the control group at various times following the rollers being taken out:
first measurement immediately after the rollers being taken out (t=0)
then at t=30, 90 and 5 hours

In order to carry out these measurements, the locks of hair are hung in an apparatus which automatically determines their length.

The activity on maintaining the curls in the hair is assessed using the parameter referred to as “curl retention” and written as CR, expressed in %, such that:

$$CR = \frac{{l}_{f} - {l}_{ti}}{{l}_{f} - {l}_{0}} \times 100$$

where CR=Curl Retention (%)
I_{f}=initial length of the uncurled lock of hair (cm)
Iₜᵢ=length of the lock of hair at time tᵢ (cm)
Iₜ₀=length of the lock of hair at time t=0 (cm)

### Results:

TABLE 1
Lengths of locks of hair and the parameter CR obtained at
T0, 30, 90 and minutes after the rollers being taken out.

| | | t = before | | | | |
|---|---|---|---|---|---|---|
| | | treatment | t 0 | t 30 mins. | t 90 mins. | t 300 mins. |
| Treated | Mean length | 216 mm | 99.91 ± 5.92 | 117.16 ± 5.52 | 127.99 ± 4.82 | 134.58 ± 5.52 |
| group | (mm) | | | | | |
| | Mean CR (%) | — | 100% | 85% | 76% | 70% |
| Control | Mean length | 216 mm | 109.67 ± 5.56 | 132.17 ± 5.85 | 145.70 ± 4.22 | 153.43 ± 3.46 |
| group | (mm) | | | | | |
| | Mean CR (%) | — | 100% | 79% | 66% | 59% |

Analysis of the results at t 0 (immediately after the rollers are taken out) shows significantly shorter locks in the group treated with the extract of acanthus compared to the control group (Iₜ₀=109.67±5.56 mm).

This difference reflects the curling effect of the acanthus (p=0.02).

Analysis of the results also shows significantly greater maintenance of the curls at t 30, t 90 and t 300 minutes in the group treated with the extract of acanthus compared to the control group (p =0.002 at t 30 and p<0.001 at t 90 and t 300).

### CONCLUSION:

Under the conditions under which this study was performed, and after a single application, the effect of the extract of acanthus on the formation and holding of the curls has been demonstrated.

1-11. (canceled)

12. A cosmetic composition comprising acanthus as active ingredient and at least one cosmetically acceptable excipient.

13. The cosmetic composition of claim 12, wherein the acanthus is in the form of an extract of acanthus, wherein the extract of acanthus exhibits a fraction of mucilages, expressed in terms of total reducing sugars, of between 1% and 80% by weight.

14. The cosmetic composition of claim 13, comprising an amount of dry extract of acanthus of between 0.01 g and 2 g per 100 g of the cosmetic composition.

15. The cosmetic composition of claim 13, wherein the amount of dry extract of acanthus is between 0.1 g and 0.7 g per 100 g of the cosmetic composition.

16. The cosmetic composition of claim 12, which is in the form of a shampoo, balm, gel, lotion, mousse, spray or cream.

17. The cosmetic composition of claim 12, wherein the acanthus is selected from Acanthus mollis, Acanthus ilicifolius, Acanthus ebracteatus, Acanthus montanus, Acanthus arboreus, Acanthus dioscoridis, Acanthus hungaricus, Acanthus sennii, Acanthus spinosus, Acanthus syriacus and Acanthus carduaceus.

18. A method for improving the creation of curls and/or maintaining a curling effect on hair comprising applying the cosmetic composition of claim 12 to the hair.

19. The method of claim 18, wherein the cosmetic composition comprises acanthus, wherein the acanthus is in the form of an extract of acanthus exhibiting a fraction of mucilages, expressed in terms of total reducing sugars, of more than 30% by weight.

20. The method of claim 18, wherein the cosmetic composition comprises acanthus, wherein the acanthus is in the form of an extract of acanthus exhibiting a fraction of mucilages, expressed in terms of total reducing sugars, of between 1% and 80% by weight.

21. The method of claim 18, wherein the cosmetic composition comprises an extract of acanthus, wherein the extract of acanthus further comprises amino acids and proteins.

22. The method of claim 18, wherein the cosmetic composition comprises acanthus, wherein the acanthus is selected from Acanthus mollis, Acanthus ilicifolius, Acanthus ebracteatus, Acanthus montanus, Acanthus arboreus, Acanthus dioscoridis, Acanthus hungaricus, Acanthus sennii, Acanthus spinosus, Acanthus syriacus and Acanthus carduaceus.
